(19) United States
(12) Patent Application Publication
(10) Pub. No.: US 2007/0238804 A1
(43) Pub. Date: Oct. 11, 2007

(54) UV-curable hard coating compositions

(76) Inventors: Encai Ho, Woodbury, MN (US); Brij Singh, North Royalton, OH (US)

Correspondence Address: HUDAK, SHUNK & FARINE, CO., L.P.A., 2020 FRONT STREET, SUITE 307, CUYAHOGA FALLS, OH, 44221, US

(21) Appl. No.: 11/784,952

(22) Filed: Apr. 10, 2007

Related U.S. Application Data
(60) Provisional application No. 60/790,969, filed on Apr. 11, 2006.

(51) Int. Cl. A61K 6/083

(52) U.S. Cl. 522/77

(57) ABSTRACT

UV curable coating compositions containing a surface modified (functionalized) colloidal silica, an acrylic monomer, an epoxy monomer and a catalytic amount of a UV polymerization initiator that are essentially-free of water are described. The compositions can optionally include a wetting agent. To form the UV curable coating compositions, an acrylic monomer, epoxy monomer and an acrylate/vinyl silane-modified colloidal silica are mixed in an aqueous/organic solvent system to form a mixture. The method features unique solvent removal steps. Solvent is first removed from the mixture under vacuum until a gel begins to form. Once gel begins to form, solvent removal is stopped and a water miscible organic solvent is added to the mixture, thus redispersing the mixture along with any formed gel. After the mixture and any formed gel have been redispersed, the remaining solvent is removed under vacuum to form a coating composition that is gel free, optically clear and essentially-free of water. A catalytic amount of the UV polymerization initiator is added to the coating composition to facilitate curing.

### CROSS REFERENCE

This application claims the priority date of U.S. Provisional Application Ser. No. 60/790,969, filed on Apr. 11, 2006, incorporated herein by reference.

### TECHNICAL FIELD

This invention is directed to UV curable coating compositions for use as a protective layer on a variety of plastic substrates, such as ophthalmic lenses, polycarbonate panels, CR-39 panels, polystyrene and others. More particularly, this invention is directed to UV curable coating compositions that are hybrid inorganic/organic nanomaterials comprising: colloidal silica nanoparticles, which have been surface-modified (functionalized) with a mixture of at least one vinyl silane and at least one acrylate silane, further combined with multifunctional epoxy and acrylic monomers. This invention is also directed to a process for making UV curable coating compositions that are essentially water free.

### BACKGROUND

Surfaces of plastic or glass-based substrates, such as ophthalmic or eyeglass lenses, are typically vulnerable to scratching, which can limit their intended function. Plastic lenses are more vulnerable to scratching and abrasion than their traditional glass counterparts. Excessive scratching results in plastic lenses being rendered unusable or, at a minimum, distracting to the user. Various coatings have been developed that, when applied to the surface of ophthalmic lenses, provide a protective layer that helps to minimize damage from scratching or other exposure. In general, a coating material comprised of polymeric material is applied to the surface of the lens or panel and is cured to form a hard surface coating resistant to scratching.

Conventional hard coatings for plastic substrates, such as ophthalmic lenses, panels, or other optical structures are well known in the art. For example, U.S. Pat. No. 4,211,823 to Suzuki et al. discloses a tintable coating composition containing fine silica particles and the hydrolysates of epoxysilanes. The compositions also contain solvents and are thermally cured, rather than by UV radiation.

U.S. Pat. No. 4,348,462 to Chung discloses a radiation curable coating composition comprising acryloxy and/or glycidoxy silanes and non-silyl acrylates in combination with silica, but the composition is not solvent free. In addition, no cross-linking agents are added to form bonds between the acrylic and glycidyl groups.

U.S. Pat No. 5,221,560 to Perkins et al. discloses a composition which includes colloidal silica and the hydrolysis products of acryloxy and/or glycidoxysilanes along with a non-silane, polymerizable functional ether. The composition, however, is not solvent free and does not include cross linking agents.

U.S. Pat. No. 6,780,232 to Treadway discloses a UV-curable coating composition that is substantially free of volatile solvents, yet retains a low viscosity. The formulation contains hydrolyzed alkoxy silanes, but is otherwise intentionally free of silica and other colloids. The patent also claims a substantial improvement in tintability of the coatings. Similarly, U.S. Pat. No. 6,100,313 to Treadway discloses a UV-curable hard coating composition, which is substantially free of solvents. It is also preferably free of silica.

U.S. Pat. No. 5,907,000 to Treadway discloses a similar refractive-index matching coating, which contains UV-curable materials; however, this formulation still contains solvents.

U.S. Pat. No. 6,250,760 to Treadway discloses a silane-based coating composition in which the addition of silica colloids at about 10% by weight, to the formulation described in U.S. Pat. No. 5,866,262 (Galic et al.) prevents the requirement of “bodying” the composition, while at the same time avoiding mudcracking defects from appearing in the coating as it cures.

The conventional coatings discussed above have some disadvantages in that the degree of hardness obtained and, hence, the protection available, is limited by the materials used to produce the coatings. Furthermore, the applicability to multiple substrates is lacking. Further, formulations containing solvents are less preferred due to concerns for worker health and safety and environmental impact. In addition, thermosetting compositions require more energy to cure than a UV-curable composition.

It has been discovered that by combining colloidal silica with functional groups from two different silanes, in particular vinyl silanes and acrylate silanes, a combination heretofore not used in the art, to form an acrylate/vinyl silane-modified silica, followed by combining the acrylate/vinyl silane-modified silica so formed with both an acrylic monomer and an epoxy monomer, a new hard coating can be formed, which, when cured, will have vastly improved scratch resistance and improved adhesion to a variety of substrates than coatings previously known in the art. In addition, a new process has been discovered, which overcomes gel formation typically encountered during the solvent removal process. The new process features unique solvent removal steps, which allow for complete removal of solvents to generate hard coatings comprising 100% solids, i.e., without water or other organic solvents. In addition, the new coatings permit further application of an anti-reflective coating on top of the hard coating.

The hard coating formulations of the invention overcome the disadvantages attributed to prior art coatings, in particular, reduced hardness/scratch resistance and poor adhesion to a variety of substrates. They are also economical to produce. In addition, because the inventive compositions are 100% solids (without water or other organic solvents), no volatiles are given off before or during the curing step.

### SUMMARY

The UV curable coating compositions described herein comprise an acrylate/vinyl silane-modified colloidal silica, an acrylic monomer, an epoxy monomer, and a catalytic amount of a UV polymerization initiator. The colloidal silica is preferably surface-modified (functionalized) by mixing it with a combination of both a vinyl silane and an acrylate silane, such as for example vinyltrimethoxysilane and 3-methacryloxypropyltrimethoxysilane. The acrylate/vinyl silane-modified colloidal silica is then combined with both an epoxy monomer and an acrylic monomer to form a clear gel free liquid coating. A photoinitiator is added to the clear gel free coating.

The UV curable coating compositions of the invention can be coated on a substrate and then cured by UV light. Coatings formed by exposing the UV curable coating composition to UV light have excellent hardness and durability, excellent adhesion to a wide variety of substrates, and excellent clarity.

One embodiment of the invention is a solvent-free, UV-curable coating composition, comprising:
a. a colloidal silica nanoparticle, which is surface-modified (functionalized) with a mixture comprising at least one vinyl silane and at least one acrylate silane;
b. at least one acrylic monomer;
c. at least one epoxy monomer; and
d. a polymerization initiator.

The UV curable coating composition of the invention is free of water and other solvents. Optionally, the UV curable coating of the invention may comprise other components, such as wetting agents and tints.

Another embodiment of this invention is directed to a method for forming a UV curable coating composition comprising the steps of:
a) preparing an acrylate/vinyl silane-modified colloidal silica by mixing at least two silanes, one of which is an acrylate and the other which is a vinyl silane, with colloidal silica;
b) mixing at least one acrylic monomer with the acrylate/vinyl silane-modified colloidal silica in an aqueous/organic solvent system to form an acrylic-silica mixture;
c) removing solvent from the acrylic-silica mixture under vacuum until a gel begins to form;
d) adding a water miscible organic solvent to the acrylic-silica mixture to redisperse the mixture in solution;
e) removing the remaining solvent under vacuum to form a clear gel free liquid acrylic-silica mixture that is essentially free of water;
f) adding at least one multi-functional epoxy monomer and at least one acrylic monomer to the clear gel free liquid acrylic-silica mixture to form a UV-curable coating composition; and
g) adding a catalytic amount of a UV polymerization initiator to the UV-curable coating composition.

### DETAILED DESCRIPTION OF THE INVENTION

The UV curable coating compositions of the present invention comprise an acrylate/vinyl silane-modified colloidal silica, an acrylic monomer, an epoxy monomer, and a catalytic amount of a UV polymerization initiator. The acrylate/vinyl silane-modified colloidal silica provides scratch resistance, and the addition of multifunctional epoxy and acrylic monomers provide improved adhesion to substrates. The epoxy monomer, in particular, provides adhesion to non-polycarbonate substrates, such as CR-39. CR-39 is trademark of PPG Industries, Inc., for allyl diglycol carbonate monomer or diethylene glycol bis (allyl carbonate) resin. The coating compositions are essentially free of water and other solvents and are thus referred to as 100% solids, i.e., no volatiles are given off before or during the curing step and 100% of the coating composition is converted to a solid film upon curing.

The novel process for making the UV curable coating compositions features unique solvent removing steps, which involve first removing solvent from a mixture of the acrylate/vinyl silane-modified colloidal silica and an acrylic monomer, under vacuum, until a gel begins to form; redispersing the mixture and any formed gel in a water miscible organic solvent; and then removing all the remaining solvent under vacuum to form a clear gel free liquid. At least one epoxy monomer and at least one acrylic monomer are added to the clear gel free liquid to form the UV-curable coating composition. Generally, the order of addition is as set forth in steps (a)-(g) above; however, some minor variation in order is permitted as long as the resulting composition is essentially free of water. The process results in coatings which comprise 100% solids as described herein.

As used herein, the term “colloidal silica” represents a broad range of silica dispersions and is not intended to be limited to a classical definition of colloid such as, e.g., particles which lie in the range from one millimicron to one micron. The term “colloidal silica” is intended to represent a wide variety of forms of finely divided, submicron-sized SiO₂ particles, i.e., nanoparticles, which can be utilized to form the coating compositions described herein, without the necessity of undue experimentation. Useful colloidal silica dispersions may be formed and maintained in aqueous or other solvent systems, and all forms are within the spirit and scope of the invention. Colloidal silica can form a polysiloxane backbone, which provides the advantages inherent in silicon products, such as wide-ranging resistance to environmental extremes.

As used herein, the phrase “essentially free of water” means that the composition does not contain an amount of water that would impact the molecular structure of the composition upon curing, although a trace amount of water may be present. A trace amount of water must be low enough so that no gel is present in the final coating composition.

As used herein, the term “100% solids” means that the compositions do not contain any water or other organic solvents, such that no volatiles are given off before or during the curing step, and 100% of the composition is converted to a solid film upon curing.

Dispersions of colloidal silica are commercially available from chemical manufacturers such as Nalco Chemical Company (Chicago, Ill.) in either acidic or basic form. An example of a commercially available colloidal silica particularly useful in the examples of the invention is NALCO™ 1034A available from Nalco Chemical Company. NALCO™ 1034A is a high purity, acidic pH aqueous colloidal silica dispersion having an average particles size of 20 nm and a SiO₂ content of approximately 34 percent by weight. Other useful colloidal silica products include NALCO™ 1030 and 1130 and Ludox™ AM-30 and AS-30 (Grace-Davison). As used in the examples, the weight in grams of the colloidal silica used includes its aqueous medium. Thus, for example, 600 grams of NALCO™ 1034A colloidal silica represents approximately 204 grams of SiO₂ by weight.

Colloidal silica modification (functionalization) using acrylate silanes is known in the art. Organic solvents including, but not limited to, isopropyl alcohol are used conventionally to form a colloidal silica solution. Modification of the colloidal silica generally is achieved by slowly adding an acrylate silane or mixture of acrylate silanes to the colloidal silica solution. The addition of acrylate silanes can be done with mixing, for example, by stirring or ultrasonic agitation. Mixing can continue, for example, for between about 0.1 hours to about 10 hours after the addition of the acrylate silanes is complete. Mixing can also be accompanied by heating. If the solution is heated, the solution can be cooled to ambient temperature after the mixing period is complete.

In the present invention, colloidal silicas are modified using a combination of acrylate and vinyl silanes to promote efficient mixing and dispersion of the silica within acrylic polymer matrices. Useful acrylate silanes are acryloxy-functional silanes having the general formula (I):

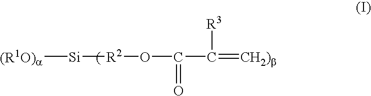

Where R¹ is a monovalent hydrocarbon radical, including halogenated radical species. R¹ can be, for example, an alkyl radical such as methyl, ethyl, propyl, butyl, etc., but may also be other saturated and unsaturated species including vinyl, aryl, etc. R² is a divalent hydrocarbon radical having from 2 to 8 carbon atoms. R³ is a hydrogen or a monovalent hydrocarbon radical. Subscript α is an integer from 1 to about 3, and subscript β is an integer from about 1 to about 3. The sum of subscripts α and β equals 4. In many cases, useful acrylate silanes have structures where α is 3 and β is 1.

Specific examples of acrylate silanes include compositions such as 3-methacryloxypropyltrimethoxysilane, 3-acryloxypropyltrimethoxysilane, 2-methacryloxy-ethyltrimethoxysilane, 2-acryloxyethyltri-methoxysilane, 3-methacryloxypropyltriethoxy-silane, 3-acryloxypropyltriethoxysilane, and 2-acryloxyethyltriethoxysilane.

Useful vinyl silanes include compositions such as vinyltrimethoxysilane, ethylene vinyl silane, ethylene vinyl acetate vinyl silane, and mixtures thereof, among others known to those skilled in the art.

For purposes of the invention, mixtures of two or more silanes are preferable for modifying the colloidal silica, and the mixture should include at least one vinyl silane and at least one acrylate silane. Most preferably, one of the vinyl silanes is vinyltrimethoxysilane, and one of the acrylate silanes is 3-methacryloxypropyltri-methoxysilane.

Once formed, the amount of acrylate/vinyl silane-modified colloidal silica used in the UV curable coating compositions of the invention varies from about 15% to about 45% by weight. Preferably, the amount of acrylate/vinyl silane-modified colloidal silica used in the coating compositions can vary from about 25% to about 35% by weight.

Once the modified colloidal silica is formed, it is mixed with at least one acrylic monomer to form an acrylic-silica mixture. Acrylic monomers useful in the coating compositions of the invention are polyfunctional acrylate ester monomers. These polyfunctional acrylate ester monomers are represented by the general formula (2):

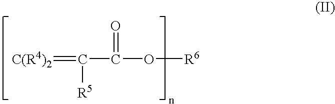

Where n is an integer from 1 to about 4; R⁴ is a C(₁₋₈) alkyl radical, R⁵ is hydrogen or a C(₁₋₈) alkyl radical; and R⁶ is a polyvalent organic radical. Polyfunctional acrylates useful in the coating compositions of the invention include monoacrylates, diacrylates, triacrylates, and tetraacrylates.

Examples of polyfunctional acrylates useful in the invention include, but are not limited to, ethoxylated pentaerythritol tetraacrylate, 1,6-hexanediol diacrylate, 2-hydroxyethyl acrylate, ethoxylated (6) trimethylolpropane triacrylate, diethylene glycol diacrylate, triethylene glycol diacrylate, tetraethylene glycol diacrylate, tripropylene glycol diacrylate, polyethylene glycol (400) diacrylate, glycidyl methacrylate, 2-hydroxy ethyl methacrylate, 3-hydroxypropyl acrylate, 3-hydroxypropyl methacrylate, 2-hydroxy-3-methacryloxypropyl acrylate, 2-hydroxy-3-acryloxypropyl acrylate, 2-hydroxy-3-methacryloxypropyl methacrylate, trimethylolpropane triacrylate, tetrahydrofurfuryl methacrylate, and mixtures thereof. The coating composition may contain a single type of polyfunctional acrylate monomer or a mixture of two or more polyfunctional acrylate monomers.

The amount of polyfunctional acrylates used in the UV curable coating composition of the invention varies from about 5% to about 85% by weight. Additionally, the amount of polyfunctional acrylates used in the coating composition can vary from about 5% to about 65% by weight; about 5% to about 55% by weight; about 5% to about 45% by weight; about 5% to about 35% by weight; about 5% to about 25% by weight; or about 5% to about 15% by weight.

Epoxy monomers useful in the UV curable coating compositions of the invention are epoxy-containing monomers having one, two, or more epoxy groups. Epoxy monomers are added to the coating composition, in addition to the acrylate monomers, to improve adhesion to certain substrates, such as, for example, CR-39. Examples of useful epoxy monomers include, but are not limited to, trimethylolpropane triglycidyl ether, 1,4-butanediol diglycidyl ether, ethylene glycol diglycidyl ether, cyclohexanedimethanol diglycidyl ether, polypropylene glycol diglycidyl ether, polyglycol diglycidyl ether, 1,6-hexanediol diglycidyl ether, trimethylolethane triglycidyl ether, castor oil triglycidyl ether, propoxylated glycerin triglycidyl ether, glycerol polyglycidyl ether; diglycerol polyglycidyl ether; glycerol propoxylate triglycidyl ether; trimethylolpropane triglycidyl ether; sorbitol polyglycidyl ether; poly(ethylene glycol) diglycidyl ether; poly(propylene glycol) diglycidyl ether; neopentyl glycol diglycidyl ether; N,N-diglycidyl-4-glycidyloxyaniline; N,N′-diglycidyltoluidine; diglycidyl 1,2-cyclo-hexanedicarboxylate; diglycidyl bisphenol A; a polymer of diglycidyl bisphenol A; poly(bisphenol A-co-epichlorohydrin), glycidyl end capped; diglycidyl of a hydrogenated bisphenol A propylene oxide adduct; diglycidyl ester of terephthalic acid; diglycidyl 1,2,3,6-tetrahydrophthalate; spiroglycoldiglycidyl ether; hydroquinone diglycidyl ether derivatives thereof, and mixtures thereof.

The amount of epoxy monomer used in the UV curable coating compositions of the invention varies from about 10% to about 50% by weight. Additionally, the amount of epoxy monomer used in the coating compositions can vary from about 10% to about 40% by weight, or from about 18% to about 35% by weight.

Useful UV polymerization initiators are ultraviolet light sensitive photoinitiator(s) or a blend(s) of such initiators. Radical-type initiators can be used alone, or combined with cationic photoinitiators. The radical-type catalysts cure the acryloxy functional portions of the composition, whereas the cationic-type catalysts cure the epoxy portions.

What is a “catalytic amount” of photoinitiators useful in the invention may vary. Typically, the catalysts are used in an amount of between about 0.05% and about 8% by weight. The catalyst may also be present in an amount of from about 1% to about 6% by weight, from about 2% to about 5% by weight; or from about 3% to about 4% by weight. The amount of photoinitiators is determined by their activity, the overlap between the photoinitiator activity spectrum and the available radiation source, the presence or absence of oxygen in the curing chamber, and the thickness of the polymer film to be cured.

Examples of UV polymerization catalysts useful in the UV curable coating composition(s) of the invention include, but are not limited to, benzophenone, 1-hydroxycyclohexyl phenyl ketone, triarylsulfonium hexafluorophosphate salts, 2-hydroxy-2-methyl-1,4-(1-methylvinyl) phenyl propanone, 2-hydroxy-2-methyl-1-phenyl-1-propanone, ethyl 4-(dimethylamino)benzoate, benzil dimethyl ketal, difunctional α-hydroxy ketone, trimethylbenzophenone, methylbenzophenone, acetophenone, 3′-hydroxyacetophenone, 4′-hydroxyaceto-phenone, 3-hydroxybenzophenone, 4-hydroxy-benzophenone, 2-methylbenzophenone, 3-methylbenzophenone, methylbenzoyl-formate, benzil, benzoin, derivatives thereof, and mixtures thereof.

The coating composition(s) of the invention can optionally include a wetting agent. It is well known that good wetting is a prerequisite to achieving good adhesion to substrates. A wetting agent increases the surface interaction between the coating composition and the substrate through Van der Waals forces and hydrogen bonding. Increased interaction between a coating composition and a substrate improves the adhesion of the coating composition and the substrate upon curing. Efficient wetting can be indicated by the contact angle of a drop of coating composition on a particular substrate. A contact angle of less than about 22° often indicates efficient wetting. A typical contact angle on polycarbonate substrates for a coating prepared as described herein was measured as approximately 18° (by axisymmetric drop shape analysis), which is believed to be enough to achieve efficient wetting for that substrate.

Different wetting agents, including BYK 300, BYK 306, and BYK 371, may be used in the coating formulations of the invention to assist in efficient wetting. BYK 300 is a preferred choice over other wetting agents in the formulations of the invention. Lenses coated with the BYK300 as the wetting agent have the additional advantage of allowing for a further anti-reflective coating to be applied over the top of the hard coating. Wetting agents such as BYK 300, BYK 306, and BYK 371 are commercially available from BYK-Chemie GmbH (Wessel, Germany).

Other examples of useful wetting agents include, but are not limited to, polyester modified poly(dimethylsiloxane), polyether modified poly(dimethylsiloxane), and mixtures thereof. Useful amounts of such wetting agents are in the range of from about 0.1 to about 1.2 weight percent. Additional useful amounts of such wetting agents can be in the range of about 0.1 to about 0.4 weight percent.

As described above, the UV curable coating compositions of the present invention include a mixture of acrylate/vinyl silane-modified colloidal silica, an acrylic monomer, an epoxy monomer, and a catalytic amount of a UV polymerization initiator. The coating compositions may optionally include a wetting agent. These coating compositions are “essentially free of water” and are referred to as comprising 100% solids.

One preferred embodiment for forming a UV curable coating composition is a method comprising the steps of:
a) preparing an acrylate/vinyl silane-modified colloidal silica by mixing at least two silanes, one of which is an acrylate silane and the other which is a vinyl silane, with colloidal silica;
b) mixing at least one acrylic monomer with the acrylate/vinyl silane-modified colloidal silica in an aqueous/organic solvent system to form an acrylic-silica mixture;
c) removing solvent from the acrylic-silica mixture under vacuum until a gel begins to form;
d) adding a water miscible organic solvent to the acrylic-silica mixture to redisperse the mixture, including any gel formed, in solution;
e) removing the remaining solvent under vacuum to form a clear gel free liquid acrylic-silica mixture that is essentially free of water;
f) adding at least one multi-functional epoxy monomer and at least one acrylic monomer to the clear gel free liquid acrylic-silica mixture to form a UV-curable coating composition; and
g) adding a catalytic amount of a UV polymerization initiator to the UV-curable coating composition.

In the first step (a) of the method, an acrylate/vinyl silane-modified colloidal silica is prepared as described above.

In the next step (b), at least one acrylic monomer is mixed with the acrylate/vinyl silane-modified colloidal silica in an aqueous/organic solvent system to form an acrylic-silica mixture. The “aqueous/organic solvent system” is a result of commercially available colloidal silica already being dispersed in an aqueous or organic solvent solution. The commercially used organic solvents can include, but are not limited to, isopropyl alcohol, ethanol, butanol, and mixtures thereof. Useful solvents are water miscible. The term “water miscible” as used herein is not intended to be limited to solvents that will mix with water and remain mixed indefinitely. Rather, “water miscible” is intended to mean that the organic solvent and water can be mixed together, and the two components will not immediately separate into distinct phases when mixing is stopped.

The invention features a unique two step solvent removal process, which is a key to obtaining the gel free compositions of the invention. In the third step (c) of the above method, solvent is first removed from the acrylic-silica mixture, under vacuum, until a gel begins to form. Processes and equipment for removing solvents from a mixture are well known to those skilled in the art. As an example, a rotary evaporation device can be used to remove solvent under vacuum. The term “gel” is intended to mean a portion of the acrylic-silica mixture that no longer has physical properties that are the same as the remainder of the mixture. Depending on the specific compounds present in the mixture, a visually noticeable color change may occur when a gel begins to form, or a change in viscosity may become apparent. For example, a colored or white residue may be visible in the mixtures. Additional indicators of gel formation are well known to and understood by those skilled in the art.

In the fourth step (d) of the method, a water miscible organic solvent is added to the acrylic-silica mixture, to redisperse the mixture, including any gel (residue) formed in step (c), in solution. The term “water miscible” is defined above and is intended to be applied here. Water miscible organic solvents useful in the method for making the coating composition include isopropyl alcohol, ethanol, butanol, and mixtures thereof.

In the fifth step (e) of the method, which is the second step of the solvent removal process, solvent is again removed, under vacuum, to form a coating composition that is “essentially free of water”, as defined above. Processes and equipment for removing solvents from a mixture are well known to those of skill in the art. Solvent is removed until no further solvent removal is discernable, and the remaining gel free liquid composition is essentially free of water as defined above.

Without intending to be bound by theory, it is thought that as the aqueous/organic solvent is initially removed from the acrylic-silica mixture (step (c) of the above-described method), a point is reached at which conditions are right for gel formation. These conditions appear to be related to both the volume and the distribution of the water molecules remaining in the mixture. In the method of the invention, the first solvent removal step is stopped as gel begins to form, and an additional water miscible organic solvent is added to redisperse any gel that has formed and any remaining water molecules (step (d) of the above-described method), over a greater volume of solvent. Then, as the solvent is removed again (step (e) of the above described method), the conditions that led to gel formation previously are not duplicated, and a gel does not form. What remains after solvent removal is a gel free liquid that is also essentially free of water.

In the sixth step (f) of the method, at least one epoxy monomer and at least one acrylic monomer are added to the acrylic-silica mixture, with stirring for about 10 minutes to about 3 hours. The result is a UV-curable coating composition that is composed of 100% solids.

UV polymerization initiators useful in the method of the invention are described above. Once added to the UV-curable coating composition, a catalytic amount of UV polymerization initiator can be mixed so that it is evenly distributed throughout the composition, as set forth in step (g) above. Selecting useful amounts of polymerization initiators is well within the capability of one of ordinary skill in the art.

Once the UV curable coating composition has been prepared, it may be spread onto a substrate surface, followed by curing. Examples of useful substrates include, but are not limited to, polycarbonate, various composite materials such as CR-39, and substrates that already have coatings, such as substrates with thermal-cured hard coats. Techniques for coating substrates include, but are not limited to, brushing, printing, bar coating, dip coating, spin coating, solution coating, roller coating, and spraying and are well known to one skilled in the art. Depending on the substrate, in-mold coating may also be used.

The coating compositions of the invention are curable by exposure to UV radiation. Ambient light, especially sunlight, often provides a range of wavelengths that include UV radiation. However, ambient light may not provide radiation with a high enough intensity to cure the coating composition in a useful period of time. Alternatively, UV radiation can be applied to the coating composition by the use of a specialized apparatus designed to emit UV radiation at a suitable intensity to cure the coating. An example of a suitable apparatus is the Model F300S from Fusion UV Systems (Gaithersburg, Md.) using a mercury bulb capable of 2-3 J/cm² intensity.

Once cured, the coating compositions described herein have excellent hardness and durability. One measure of the durability of a coating is the Bayer abrasion test, which is a measure of abrasion resistance. In the Bayer abrasion test, abrasive media is oscillated back and forth over the surface of a coating for a predetermined number of cycles. Once the abrasion period is complete, the haze (light scatter) of the coating is measured and compared to a haze measurement taken prior to the abrasion period. The Bayer haze value is the percentage increase in haze caused during the abrasion period. Dividing the Bayer haze value for a standard substrate (an uncoated pIano lens of CR-39 resin provided as a reference by the International Standards Organization) by the Bayer haze value for the coating yields a value called the Bayer ratio. Bayer ratios of greater than about 1.8, when comparing uncoated CR-39 lenses to sample coatings of the invention can be achieved. Further, Bayer ratios of greater than about 1.9, greater than about 2.0, greater than about 2.1, or greater than about 2.2, when comparing uncoated CR-39 lenses to sample coatings of the invention can also be achieved. The hardness and durability of a particular coating depends upon the specific composition used and the ability to alter composition formulations within the above guidelines can be useful in engineering a coating with specific qualities.

The UV curable coating compositions described herein also have excellent adhesion over a wide variety of substrates. The specific materials used in the coating composition affect the adhesion of a particular composition to a particular substrate. For example, it is known that coating compositions containing only acrylic monomers may have excellent adhesion to bare polycarbonate substrates, but poor adhesion to bare CR-39 substrates. However, it has been found that coating compositions containing combinations of both acrylic monomers and epoxy monomers have excellent adhesion to both bare polycarbonate and bare CR-39 substrates. These coating compositions also provide affordable and reliable coatings that have superior performance when compared with thermal-cured hard coats as used on bare polycarbonate lenses. Further, coating compositions containing combinations of both acrylic and epoxy monomers have excellent adhesion to substrates already coated by thermal-cured hard coats. Examples of substrates coated with a thermal-cured hard coat, to which excellent adhesion has been achieved for the coating compositions as described herein, include Poly-Orcolite (Vision-Ease Lens, Inc.; Ramsey, Minn.), Poly-Gentex (Gentex Corporation; Zeeland, Mich.), and hardcoat coated polycarbonate and CR-39 substrates from Hoya Vision Care, N.A. (Lewisville, Tex.).

If desired, an anti-reflective coating can be used on the outer, exposed side of the cured coating composition, to reduce the reflection at the surface and allow a higher level of visible light transmission. Anti-reflective coatings can also increase the durability of the coated substrate surface. Typically, anti-reflective coatings include multiple different sub-layers. The thickness of each sub-layer is often related to an even whole number division of the wavelength of light that is most preferred to be transmitted through the coated material. Anti-reflective coatings are well known in the art, and information on designing and depositing anti-reflective layers on objects can be found in such references as the Handbook of Optics (McGraw Hill, 2^{nd} Ed.), and Design of Optical Interference Coatings (McGraw Hill), which are incorporated herein by reference. Anti-reflective coatings may include sublayers of many different materials, such as, but not limited to, Al₂O₃, ZrO₃, MgF₂, SiO₂, cryolite, LiF, ThF₄, CeF₃, PbF₂ZnS, ZnSc, Cr, Si, Ge, Te, MgO, Y₂O, Sc₂O₃, SiO, HfO, HfO₂, ZrO₂, CeO₂, Nb₂O₃, Ta₂O₅, and TiO₂. Thus, an anti-reflective coating on the outer side of a coating made from the present coating composition can improve the visible light transmission level and the durability of the surface.

Additionally, the coating compositions of the invention, once cured, do not appreciably alter the refractive index of the substrate, i.e., the refractive index of the coated substrate is very close to the refractive index of the uncoated substrate. As such, the coating compositions of the invention can be used on substrates without detrimental impact on the optical qualities of the substrate. For example, the coating compositions described herein can often be used on eyeglass lenses or windows without greatly impacting their intended optical qualities. Further, many of the compositions described herein can be tinted. Other uses for the coating compositions of the invention include a wide variety of applications where abrasion resistance, good adhesion, and optical clarity are desired. Examples of applications for the coating compositions of the invention include, but are not limited to, ophthalmic lenses (such as eyeglass lenses), polycarbonate panels, automotive windshields and windows, and electronic touch panels. Based on the description and examples herein, other applications for the coating compositions of the invention are obvious.

In comparison to thermal-cured hard coats, UV curable coatings provide a fast and energy-saving coating process however, the scratch resistance of UV curable coatings needs to be improved. For ophthalmic lens application, the adhesion to different substrates and anti-reflective (AR) coatings also needs to be improved.

The following examples are intended for illustration purposes only and should not be construed as limitations upon the claims or upon the individual components useful in the claimed compositions or methods.

### EXAMPLES

The coatings of the invention are based on organic/inorganic hybrid nanomaterials, comprising multifunctional epoxy and acrylic monomers and surface-modified (functionalized) inorganic silica nanoparticles, which offer excellent abrasion and adhesion to a variety of substrates, and optical clarity. In the coating compositions described in the examples, the acrylate/vinyl silane-modified colloidal silica provided hardness and abrasion resistance; and the combination of multifunctional epoxy and acrylic monomers improved the adhesion to different substrates.

For purposes of illustration of the invention, silanes, such as vinyltrimethoxysilane (VTMS), 3-methacryloxypropyltrimethoxysilane (MPTMS), and combinations of VTMS/MPTMS, were used to modify inorganic colloidal silica nanoparticles. A combination of VTMS/MPTMS was preferred, because it achieved unexpectedly improved hardness and scratch resistance and superior adhesion over what had been known previously. After surface modification, appropriate multifunctional epoxy and acrylic monomers and wetting agents were mixed to generate optically clear coating formulations essentially free of water and having 100% solids as defined above. Photoinitiators were added to facilitate UV curing.

By taking the benefit of the unique properties of nanomaterials, such as colloidal stability and high surface area, we were able to modify (functionalize) inorganic colloidal silica (nanoparticles) using reactive vinyl and acrylate silanes, such as vinyltrimethoxysilane (VTMS) and 3-methacryloxypropyltrimethoxysilane (MPTMS), more particularly combinations of VTMS/MPTMS. The acrylate/vinyl silane-modified silica nanoparticles were further incorporated in coating systems and significantly and unexpectedly improved the scratch resistance while remaining clear. By selecting proper combinations of acrylic and epoxy multifunctional monomers, hard coatings were prepared, which unexpectedly adhered to different plastic substrates, such as CR-39, PC, polystyrene, and others.

For PC panels or lenses, VTMS/MPTMS modified colloidal silica, ethoxylated pentaerythritol tetraacrylate (SR494) and 1,6-hexanediol diacrylate (SR238) were used as a resin system. SR494 is a relatively new monomer that provides better scratch resistance than other monomers commonly used in the past.

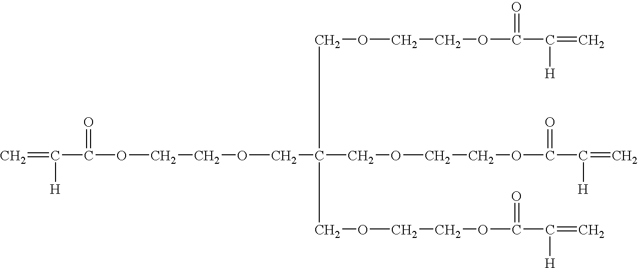

For ophthalmic applications, VTMS/MPTMS modified colloidal silica, acrylic monomers, and epoxy monomers were used as a resin system. Both free-radical and cationic-type UV polymerization were also used in the systems.

The coatings created in the examples were tested for hardness and durability using the Bayer abrasion test, for adhesion using cross-hatch adhesion testing, and the refractive index and tintability were determined. The Bayer abrasion test involves oscillating abrasive media back and forth over the surface of a coating and then determining the amount of light scatter/haze created (“haze gain”). The abrasive media used was 500 g of Alundum, and a complete test process was 600 cycles at a speed of 150 cycles/min. Dividing the haze gain value of the uncoated piano lens of CR-39 resin (provided as a reference by the International Standards Organization) by the haze gain for the sample yields a value called the Bayer ratio.

Cross-hatch adhesion is a standard procedure for evaluating the adhesion of a coating on a substrate. In this procedure, a cutting device such as a razor blade is used to make six parallel cuts about 1.5 mm (±0.5 mm) apart and approximately 15 to 20 mm in length in the coating on the substrate. Then, another six parallel cuts 1.5 mm (±0.5 mm) apart are made in the coating perpendicular to the first set. This forms a cross-hatched pattern of squares over which tape is applied, such as 3M Scotch brand #600 and 8981. The tape then is pulled rapidly as close to an angle of 180° as possible, and the percent adhesion is quantified by the amount of coating removed from the squares in the cross-hatched pattern. The 180° reference means that the tape is pulled back over itself in a direction that is nearly parallel to the lens surface.

The refractive index for the samples was measured using a Model 2010/M prism coupler from Metricon Corporation (Pennington, N.J.). The Model 2010M prism coupler provides rapid measurements of the refractive index of bulk plastics such as PC and CR-39 lenses and thin optical films such as the UV-curable hard coats with a resolution of ±0.0005.

Tintability of the coating compositions of the examples was also evaluated by immersing a coated substrate into an aqueous solution of an organic dye at about 60° C. to about 80° C. If the coating was tintable, the coating appeared colored upon removal from the organic dye solution.

Generally, the major components used in the examples, along with ranges, were as follows:
Acrylate/vinyl silane-modified colloidal silica in the range of 1545 wt. %, also 25-35 wt. %.
SR494 in the range of 5-45 wt. %, and 5-25 wt. %.
SR238 or SR306 in the range of 10-45 wt. %, also 12-35 wt. %.
2-hydroxyethyl acrylate in the range of 5-25 wt. %, also 5-15 wt. %.
ERISYS GE-30 in the range of 0-50 wt. %, also 18-35 wt. %.
SR344 in the range of 12-30 wt. %, also 15-25 wt. %.
Glycidyl methacrylate in the range of 2-15 wt. %, also 5-10 wt. %.
Benzophenone in the range of 2-8 wt. %, also 3-6 wt. %.
1-hydroxycylohexyl phenyl ketone or Esacure 100F in the range of 2-6 wt. %, also 2-4 wt. %.
Triarylsulfonium hexafluorophosphate salts 50% in propylene carbonate in the range of 0.7-8 wt. %, also 2-5 wt. %.
Wetting agents, such as BYK 371, BYK 300, BYK 306, and FC 4430 in the range of 0.1-1.2 wt. %, also 0.1-0.4 wt. %, are used.

### Example 1

The Example 1 Coating contains colloidal silica modified by a mixture of VTMS (vinyltrimethoxysilane) and MPTMS (3-methacryloxypropyltrimethoxysilane), Comparison Coating 1A (containing only VTMS modified silica), and Comparison Coating 1B (containing only MPTMS modified silica) were prepared and analyzed.

Coating Synthesis

A mixture comprising 51.2 g of MPTMS (Gelest, Inc.; Morrisville, Pa.) and 30.4 g of VTMS (Gelest, Inc.; Morrisville, Pa.) was added dropwise at room temperature to a solution of 600 g of NALCO™ 1034A silica (Nalco Chemical Company; Naperville, Ill.) in 800 g of isopropyl alcohol under rapid stirring. Stirring was continued for 15 minutes after the last dropwise addition, and then the mixture was heated to 70° C. for 3 hours under continued rapid stirring. After heating, the mixture was cooled to room temperature, and 154.6 g of ethoxylated pentaerythritol tetraacrylate (SR494, Sartomer Company, Inc., Exton, Pa.) and 246 g of 1,6-hexanediol diacrylate (SR238, Sartomer Company, Inc.; Exton, Pa.) were added with constant stirring.

Next, most of the solvents were distilled off under reduced pressure in a rotary evaporator, leaving a white residue. The white residue was then redissolved in 300 g of isopropyl alcohol. The resulting solution was then further distilled in a rotary evaporator under reduced pressure, yielding a clear gel free liquid.

69.2 g of 2-hydroxyethyl acrylate (Aldrich Chemical Co., Inc., Milwaukee, Wis.), 28.6 g of benzophenone (Aldrich Chemical Co., Inc., Milwaukee, Wis.), 14.4 g of 1-hydroxycyclohexyl phenyl ketone (Aldrich Chemical Co., Inc., Milwaukee, Wis.), and 2.4 g of BYK 371 (BYK Chemie GmbH; Wesel, Germany) were mixed with the above noted clear liquid, and the mixture was then stirred for 3 hours. A clear coating liquid was formed. The viscosity of the coating was 38 cp at 25° C.

Comparison Coatings 1A and 1B: Synthesis

Comparison Coatings 1A and 1B were prepared using the same method used to form the Example 1 Coating, with the following differences: Comparison Coating 1A comprised 60.8 g of VTMS (and no MPTMS), and Comparison Coating 1B comprised 102.4 g of MPTMS (and no VTMS).

Film Formation & Testing

Polycarbonate substrates were cleaned using high-pressure water and dried using air flow. Each coating solution was spin-coated onto individual polycarbonate substrates (lenses or panels) and cured using a 2-3 J/cm² mercury bulb (Fusion UV Systems, Inc., Gaithersburg, Md.) for two hours. After curing, the coated lenses were subjected to 600 cycles of Bayer Abrasion Testing. Additionally, an uncoated CR-39 substrate was tested as a control for comparison purposes. Several samples of each coating combination were created, and the results for each measurement were the average value for these samples.

Coating Performance

Table 1 shows the performance for the Example 1 Coating, Comparison Coating 1A, Comparison Coating 1B, and an uncoated CR-39 substrate.

TABLE 1
Example 1 Results

| | Example 1 | Comparison | Comparison | Uncoated |
|---|---|---|---|---|
| | Coating | Coating 1A | Coating 1B | CR-39 |
| Coating Thickness | 3-6 | 3-6 | 3-6 | — |
| (μm) | | | | |
| Bayer Haze (%) | 8.4 | 9.2 | 9.6 | 22-25 |
| Bayer Ratio | 2.8 | 2.56 | 2.45 | 1 |
| Cross-Hatch | 100% Pass | 100% Pass | 100% Pass | — |
| Adhesion^{a} | | | | |
| Cross-Hatch | No | — | — | — |
| Adhesion (CR-39)^{b} | | | | |
| Refractive Index | 1.51 | 1.509 | 1.51 | 1.49 |

^{a}On a bare polycarbonate substrate.
^{b}On a bare CR-39 substrate.

As shown in Table 1, the Example 1 Coating exhibited a Bayer Ratio of 2.8, indicating that the sample was a hard, abrasive-resistant coating. The VTMS/MPTMS combination used in the Example 1 Coating yielded a higher Bayer ratio (when compared with the uncoated control) than that of either Comparison Coating 1A or Comparison Coating 1B, indicating that the VTMS/MPTMS mixture provided a harder coating than using VTMS or MPTMS alone. While not wishing to be bound by theory, it is postulated that the combination of VTMS/MPTMS provides better surface coverage of silane molecules on the colloidal silica nanoparticles.

The Example 1 Coating (without epoxy monomer) would not adhere to a bare CR-39 substrate; but a drop of the coating on a bare polycarbonate substrate had a contact angle of about 18° (as measured by axisymmetric drop shape analysis), indicating efficient wetting. Additionally, each coating was tintable.

### Example 2

The Example 2 Coating comprised colloidal silica modified by a mixture of VTMS and MPTMS and was prepared and analyzed as set forth below.

Coating Synthesis

A mixture comprising 64 g of MPTMS and 38 g of VTMS was added dropwise, at room temperature, to a solution of 750 g of NALCO™ 1034A silica and 1000 g of isopropyl alcohol under rapid stirring. Stirring was continued for 15 minutes after the last dropwise addition, and then the mixture was heated to 70° C. for 3 hours under continued rapid stirring. After heating, the solution was cooled to room temperature, and 433.8 g of ethoxylated pentaerythritol tetraacrylate (SR494), 59.8 g of ethoxylated (6) trimethylolpropane triacrylate (SR499, Sartomer Company, Inc., Exton, Pa.), and 103 g of 2-hydroxyethyl acrylate (Sartomer Company, Inc., Exton, Pa.) were added with constant stirring.

Next, most of the solvents were distilled off in a rotary evaporator under reduced pressure, leaving a white residue, which indicates gel formation. The white residue was then redissolved in 450 g of isopropyl alcohol. The resulting solution was then further distilled in a rotary evaporator under reduced pressure, yielding a clear gel free liquid.

156 g of tripropylene glycol diacrylate (SR306, Sartomer Company, Inc., Exton, Pa.), 35.6 g of benzophenone, 17.8 g of 1-hydroxycyclohexyl phenyl ketone, and 2.4 g of BYK 300 (BYK Chemie GmbH; Wesel, Germany) were added to the above clear liquid, and the mixture was stirred for 3 hours. A clear coating liquid was formed. The viscosity of the coating was 122 cp at 25° C.

Film Formation & Testing

Films were formed and tested using the same methods as used in Example 1. Additional examples were created that also included an anti-reflective coating. The anti-reflective coating, in this case Cr/SiO/HfO/SiO₂/HfO/SiO₂, was applied on top of a layer of the coating composition by vacuum deposition using a B121V vacuum coating machine. The samples, including those having an anti-reflective coating, were tested for cross-hatch adhesion to the coating composition.

Coating Performance

Table 2 shows the performance of the Example 2 Coating and an uncoated CR-39 substrate.

TABLE 2
Example 2 Results

| | | Uncoated |
|---|---|---|
| | Example 2 | CR-39 |
| Coating Thickness μm) | 3-6 | — |
| Bayer Haze (%) | 10.4 | 22-25 |
| Bayer Ratio | 2.3 | 1 |
| Cross-Hatch Adhesion^{a} | 100% Pass | — |
| Cross Hatch Adhesion (CR-39)^{b} | No | — |
| Cross-Hatch Adhesion (AR) | Pass | — |
| Refractive Index | 1.506 | 1.49 |

^{a}On a bare polycarbonate substrate.
^{b}On a bare CR-39 substrate.

As shown in Table 2, the Example 2 Coating exhibited a Bayer ratio of 2.3, which indicates a hard, abrasion-resistant coating. The coating did not contain an epoxy monomer and would not adhere to a bare CR-39 substrate. Additionally, the coating was tintable.

### Example 3

The Example 3 Coating, which comprised colloidal silica modified by a mixture of VTMS and MPTMS, was prepared and coated on a substrate. An anti-reflective coating was then added, and the coated substrate was analyzed.

Coating Synthesis

A mixture containing 37.5 g of MPTMS and 22.1 g of VTMS was added dropwise at room temperature to a solution of 375 g of NALCO™ 1034A silica and 500 g of isopropyl alcohol under rapid stirring. Stirring was continued for 15 minutes after the last dropwise addition, then the mixture was heated to 70° C. for 3 hours under continued rapid stirring. After heating, the solution was cooled to room temperature, and 263.6 g of ethoxylated pentaerythritol tetraacrylate (SR494) and 40 g of ethoxylated (6) trimethylolpropane triacrylate (SR499) were added under constant stirring.

Next, most of the solvents were distilled off under reduced pressure using a rotary evaporator, leaving a white residue. The white residue was then redissolved in 230 g of isopropyl alcohol. The solution was then evaporated in a rotary evaporator under reduced pressure, yielding a clear gel free liquid.

94 g of tripropylene glycol diacrylate (SR306), 23.3 g of benzophenone, 11.6 g of 1-hydroxycyclehexyl phenyl ketone, and 2.4 g of BYK 300 was added to the above clear liquid, and the mixture was stirred for 3 hours. A clear coating liquid was formed.

Film Formation & Testing

Film examples were formed and tested using the same method as used in Example 2. Additionally, the samples, including one having an anti-reflective coating, were subjected to Bayer abrasion testing.

Coating Performance

Table 3 shows the performance of the Example 3 Coating, the Example 3 +anti-reflective coating, and an uncoated CR-39 substrate.

TABLE 3
Example 3 Results

| | | Example | Uncoated |
|---|---|---|---|
| | Example 3 | 3 + AR | CR-39 |
| Coating Thickness (μm) | 3-6 | — | — |
| Bayer Haze (%) | 11.2 | 7.56 | 22.25 |
| Bayer Ratio | 2.0 | 3.0 | 1 |
| Cross-Hatch Adhesion (PC)^{a} | 100% Pass | — | — |
| Cross-Hatch Adhesion (CR-39)^{b} | No | — | — |
| Cross-Hatch Adhesion (AR) | — | 100% Pass | — |
| Refractive Index | 1.51 | — | 1.49 |

^{a}On a bare polycarbonate substrate.
^{b}On a bare CR-39 substrate.

As shown in Table 3, the Example 3 Coating alone exhibited a Bayer ratio of 2.0 and, with an additional anti-reflective coating, a Bayer ratio of 3.0. These high Bayer ratios indicated hard, abrasion-resistant coatings. The improvement in Bayer ratio between the Example 3 Coating and the Example 3 + anti-reflective coating indicated that the Example 3 Coating has a very good compatibility with the anti-reflective coating. The Example 3 Coating, which did not contain an epoxy monomer, would not adhere to a bare CR-39 substrate. Additionally, the Example 3 Coating was tintable.

### Example 4

The Example 4 Coating, which contained colloidal silica modified by a mixture of VTMS and MPTMS, was prepared and analyzed as set forth below.

Coating Synthesis

A mixture containing 105 g of MPTMS and 62.6 g of VTMS was added slowly to a solution of 1235.5 g of NALCO™ 1034A silica and 1650 g of isopropyl alcohol under rapid stirring. Stirring was continued for 15 minutes after the addition. The mixture was then heated to 70° C. for 3 hours under continued rapid stirring. After heating, the solution was cooled to room temperature, and 894 g of ethoxylated pentaerythritol tetraacrylate (SR494) and 370 g of 1,6-hexanediol diacrylate (SR238) were added under constant stirring.

Next, most of the solvents were distilled off under reduced pressure using a rotary evaporator, leaving a white residue. The white residue was then redissolved in 300 g of isopropyl alcohol. The solution was then distilled using a rotary evaporator under reduced pressure, yielding a clear gel free liquid.

72.5 g of benzophenone, 41.2 g of 1-hydroxycyclohexyl phenyl ketone, 57 g of ethoxylated pentaerythritol tetraacrylate (SR494), 50 g of 1,6-hexanediol diacrylate (SR238), and 2.8 g of BYK 371 were added to the above clear liquid, and the mixture was stirred for 3 hours. A clear coating liquid was formed.

Film Formation & Testing

A film was formed and tested using the same method as used in Example 2.

Coating Performance

Table 4 shows the performance for the Example 4 Coating and an uncoated CR-39 substrate.

TABLE 4
Example 4 Results

| | | Uncoated |
|---|---|---|
| | Example 4 | CR-39 |
| Coating Thickness μm) | 3-6 | — |
| Bayer Haze (%) | 11.0 | 22-25 |
| Bayer Ratio | 2.10 | 1 |
| Cross-Hatch Adhesion (PC)^{a} | 100% Pass | — |
| Cross Hatch Adhesion (CR-39)^{b} | No | — |
| Cross-Hatch Adhesion (AR) | 100% Pass | — |
| Refractive Index | 1.51 | 1.49 |

^{a}On a bare polycarbonate substrate.
^{b}On a bare CR-39 substrate.

As shown in Table 4, the Example 4 Coating exhibited a Bayer ratio of 2.1. This high Bayer ratio indicated that the coating is hard and abrasion-resistant. The coating would not adhere to a bare CR-39 substrate. Additionally, the coating was tintable.

### Example 5

The Example 5 Coating, which contained colloidal silica modified by a mixture of VTMS and MPTMS, was prepared and analyzed. The coating had good adhesion to CR-39 substrates, but poor adhesion to bare polycarbonate substrates.

Coating Synthesis

A mixture containing 26.8 g of MPTMS and 16 g of VTMS was added dropwise at room temperature to a solution of 316 g of NALCO™ 1034A silica and 400 g of isopropyl alcohol under rapid stirring. Stirring was continued for 15 minutes after the last dropwise addition, then the mixture was heated to 70° C. for 3 hours under continued rapid stirring. After heating, the solution was cooled to room temperature, and 43 g of ethoxylated pentaerythritol tetraacrylate (SR494), 43 g of polyethylene glycol (400) diacrylate (SR344 Sartomer Company, Inc.; Exton, Pa.), and 36 g of 2-hydroxyethyl acrylate were added with constant stirring.

Next, most of the solvents were distilled off under reduced pressure using a rotary evaporator, leaving a residue. The residue was then redissolved in 300 g of isopropyl alcohol. The solution was then distilled in a rotary evaporator under reduced pressure, yielding a clear gel free liquid.

84 g of trimethylolpropane triglycidyl ether (ERISYS GE-30, CVC Specialty Chemicals, Inc.; Moorestown, N.J.) (an epoxy), 16.0 g of glycidyl methacrylate (Aldrich Chemical Co., Inc.; Milwaukee, Wis.) (an epoxy) and 60.0 g of polyethylene glycol (400) diacrylate (SR344) were added to the above clear liquid under rapid stirring. Next, 16.8 g of benzophenone, 8.6 g of Esacure 100F (Sartomer Company, Inc.; Exton, Pa.), and 21 g of triarylsulfonium hexafluorophosphate salts-50% in propylene carbonate (Aldrich Chemical Co., Inc.; Milwaukee, Wis.) and 2.6 g of BYK 300 were added to the liquid composition, which was then stirred for 3 hours. A clear coating liquid was formed. The viscosity of the coating was 61.2 cp at 25° C.

Film Formation & Testing

Example 5 included an analysis of adhesion to a CR-39 substrate. The CR-39 substrates were cleaned using caustic solution prior to film deposition. Washing of plain (unsurfaced) CR39 lenses with a caustic solution was found to improve adhesion. This step was not required for surfaced CR39 lenses, in which washing with pressurized water was sufficient. Otherwise, films were formed and tested using the same method as used in Example 2.

Coating Performance

Table 5 shows the performance of the Example 5 Coating and an uncoated CR-39 substrate.

TABLE 5
Example 5 Results

| | | Uncoated |
|---|---|---|
| | Example 5 | CR-39 |
| Coating Thickness (μm) | 3-6 | n/a |
| Bayer Haze (%) | 11.6 | 24.6 |
| Bayer Ratio | 2.08 | 1 |
| Cross-Hatch Adhesion (PC)^{a} | poor | — |
| Cross-Hatch Adhesion (CR-39)^{b} | 100% Pass | — |
| Cross-Hatch Adhesion (AR) | 100% Pass | — |
| Refractive Index | 1.51 | 1.49 |

^{a}On a bare polycarbonate substrate.
^{b}On a bare CR-39 substrate.

As shown in Table 5, the Example 5 Coating exhibited a Bayer ratio of 2.08. This high Bayer ratio indicated that the coating was hard and abrasion-resistant. The coating exhibited good adhesion to a bare uncoated CR-39 substrate, but had poor adhesion to a polycarbonate substrate. Additionally, the coating was tintable.

### Example 6

The Example 6 Coating, which contained colloidal silica modified by a mixture of VTMS and MPTMS, was prepared and analyzed. The coating had good adhesion to both CR-39 substrates and bare polycarbonate substrates.

Coating Synthesis

A mixture containing 51.2 g of MPTMS and 30.4 g of VTMS was added dropwise at room temperature to a solution of 600 g of NALCO™ 1034A silica and 800 g of isopropyl alcohol under rapid stirring. Stirring was continued for 15 minutes after the last dropwise addition, then the mixture was heated to 70° C. for 3 hours under continued rapid stirring. After heating, the solution was cooled to room temperature, and 307.2 g of ethoxylated pentaerythritol tetraacrylate (SR494) and 128 g of 1,6-hexanediol diacrylate (SR238) were added under constant stirring.

Next, most of the solvents were distilled off under reduced pressure using a rotary evaporator, leaving a white residue. The white residue was then redissolved in 300 g of isopropyl alcohol. All of the solvents were then removed using the rotary evaporator under reduced pressure, yielding a clear gel free liquid.

76.8 g of 2-hydroxyethyl acrylate and 162.2 g of trimethylolpropane triglycidyl ether (ERISYS GE-30) were added to the above clear liquid under rapid stirring. Then 28.6 g of benzophenone, 14.4 g of 1-hydroxycyclohexyl phenyl ketone, 17.8 g of triarylsulfonium hexafluorophosphate salts-50% in propylene carbonate, and 2.4 g of BYK 300 were mixed with the liquid composition, and the mixture was stirred for 3 hours. A clear coating liquid was formed. The viscosity of the coating was 135 cp at 25° C.

Film Formation & Testing

Films were formed and tested using the same method as used in Example 5. Additionally, the samples, including one having an anti-reflective coating, were subjected to Bayer abrasion testing.

Coating Performance

Table 6 shows the performance of the Example 6 Coating, the Example 6 plus anti-reflective coating, and an uncoated CR-39 substrate.

TABLE 6
Example 6 Results

| | | Example | Uncoated |
|---|---|---|---|
| | Example 6 | 6 + AR | CR-39 |
| Coating Thickness (μm) | 3-6 | — | — |
| Bayer Haze (%) | 10.6 | 7.6 | 22.6 |
| Bayer Ratio | 2.13 | 2.97 | 1 |
| Cross-Hatch Adhesion (PC)^{a} | 100% Pass | 100% Pass | — |
| Cross-Hatch Adhesion (CR-39)^{b} | 100% Pass | — | — |
| Cross-Hatch Adhesion (AR) | — | 100% Pass | — |
| Refractive Index | 1.51 | — | 1.49 |

^{a}On a bare polycarbonate substrate.
^{b}On a bare CR-39 substrate.

As shown in Table 6, the Example 6 Coating alone exhibited a Bayer ratio of 2.13, and with an addition of anti-reflective coating, exhibited a Bayer ratio of 2.97. These high Bayer ratios indicated hard, abrasion-resistant coatings. The improvement in Bayer ratio between the Example 6 Coating and the Example 6 plus anti-reflective coating, indicated that the Example 6 Coating had a very good compatibility with the anti-reflective coating. The Example 6 Coating exhibited adhesion to both a bare CR-39 substrate and a polycarbonate substrate. Additionally, the coating was tintable.

### Example 7

The Example 7 Coating, which contained colloidal silica modified by a mixture of VTMS and MPTMS, was prepared and analyzed. The coating had good adhesion to both CR-39 substrates and bare polycarbonate substrates.

Coating Synthesis

A mixture containing 53.6 g of MPTMS and 31.95 g of VTMS was added dropwise at room temperature to a solution of 632 g of NALCO™ 1034A silica and 800 g of isopropyl alcohol under rapid stirring. Stirring was continued for 15 minutes after the last addition, then the mixture was heated to 70° C. for 3 hours under continued rapid stirring. After heating, the solution was cooled to room temperature, and 61.8 g of ethoxylated pentaerythritol tetraacrylate (SR494), 128.8 g of 1,6-hexanediol diacrylate (SR238), and 87.6 g of 2-hydroxyethyl acrylate were added with constant stirring.

Next, most of the solvents were distilled off under reduced pressure using a rotary evaporator, leaving a white residue. The white residue was then redissolved in 300 g of isopropyl alcohol. All of the solvents were then removed using a rotary evaporator under reduced pressure, yielding a clear gel free liquid.

205 g of trimethylolpropane triglycidyl ether (ERISYS GE-30), 38.6 g of glycidyl methacrylate, and 228 g of polyethylene glycol (400) diacrylate (SR344) were added to the above clear liquid under rapid stirring. Then, 32.6 g of benzophenone, 16.8 g of Esacure 100F, 21 g of triarylsulfonium hexafluorophosphate salts-50% in propylene carbonate, and 2.6 g of BYK 300 were added, and the mixture was stirred for 3 hours. A clear coating liquid was formed. The viscosity of the coating was 82.6 cp at 25° C.

Film Formation & Testing

Films were formed and tested using the same method as used in Example 5.

Coating Performance

Table 7 shows the performance of the Example 7 Coating and an uncoated CR-39 substrate.

TABLE 7
Example 7 Results

| | | Uncoated |
|---|---|---|
| | Example 7 | CR-39 |
| Coating Thickness (μm) | 3-6 | n/a |
| Bayer Haze (%) | 11.6 | 22.7 |
| Bayer Ratio | 1.96 | 1 |
| Cross-Hatch Adhesion (PC)^{a} | 100% Pass | — |
| Cross-Hatch Adhesion (CR-39)^{b} | 100% Pass | — |
| Cross-Hatch Adhesion (AR) | 100% Pass | — |
| Refractive Index | 1.51 | 1.49 |

^{a}On a bare polycarbonate substrate.
^{b}On a bare CR-39 substrate.

As shown in Table 7, the Example 7 Coating exhibited a Bayer ratio of 1.96. This high Bayer ratio indicated that the coating was hard and abrasion-resistant. The coating exhibited adhesion to both a bare CR-39 substrate and a polycarbonate substrate and was tintable.

### Example 8

The Example 8 Coating, which contained colloidal silica modified by a mixture of VTMS and MPTMS, was prepared and analyzed. The coating had good adhesion to both CR-39 substrates and bare polycarbonate substrates.

Coating Synthesis

A mixture containing 53.6 g of MPTMS and 31.95 g of VTMS was added dropwise at room temperature to a solution of 632 g of NALCO™ 1034A silica and 800 g of isopropyl alcohol under rapid stirring. Stirring was continued for 15 minutes after the last addition, then the mixture was heated to 70° C. for 3 hours under continued rapid stirring. After heating, the solution was cooled to room temperature, and 61.8 g of ethoxylated pentaerythritol tetraacrylate (SR494), 128.8 g of 1,6-hexanediol diacrylate (SR238), and 125.6 g of 2-hydroxyethyl acrylate were added with constant stirring.

Next, most of the solvents were distilled off under reduced pressure using a rotary evaporator, leaving a residue. The residue was then redissolved in 300 g of isopropyl alcohol. All of the solvents were removed using a rotary evaporator under reduced pressure, yielding a clear gel free liquid.

205 g of trimethylolpropane triglycidyl ether (ERISYS GE-30), 38.6 g of glycidyl methacrylate, and 192 g of polyethylene glycol (400) diacrylate (SR344) were added to the above clear liquid under rapid stirring. Then, 32.6 g of benzophenone, 16.8 g of 1-hydroxycyclohexyl phenyl ketone, 11 g of triarylsulfonium hexa-fluorophosphate salts-50% in propylene carbonate, and 1.36 g of BYK 300 were added, and the mixture was stirred for 3 hours. A clear coating liquid was formed. The viscosity of the coating was 90.7 cp at 25° C.

Film Formation & Testing

Films were formed and tested using the same method as used in Example 5.

Coating Performance

Table 8 shows the performance of the Example 8 Coating and an uncoated CR-39 substrate.

TABLE 8
Example 8 Results

| | | Uncoated |
|---|---|---|
| | Example 8 | CR-39 |
| Coating Thickness (μm) | 3-6 | n/a |
| Bayer Haze (%) | 11.4 | 22.7 |
| Bayer Ratio | 1.99 | 1 |
| Cross-Hatch Adhesion (PC)^{a} | 100% Pass | — |
| Cross-Hatch Adhesion (CR-39)^{b} | 100% Pass | — |
| Cross-Hatch Adhesion (AR) | 100% Pass | — |
| Refractive Index | 1.509 | 1.49 |

^{a}On a bare polycarbonate substrate.
^{b}On a bare CR-39 substrate.

As shown in Table 8, the Example 8 Coating exhibited a Bayer ratio of 1.99. This high Bayer ratio indicated that the coating was hard and abrasion-resistant. The coating exhibited adhesion to both a bare CR-39 substrate and a polycarbonate substrate and was tintable.

### Example 9

The Example 9 Coating, which contained colloidal silica modified by a mixture of VTMS and MPTMS, was prepared and analyzed. This coating had good adhesion to CR-39 substrates, but exhibited poor adhesion to bare polycarbonate substrates. This coating also exhibited good adhesion to commercially available polycarbonate and CR-39 substrates that were coated with thermally cured hard coats.

Coating Synthesis

A mixture containing 18 g of MPTMS and 10.6 g of VTMS was added dropwise at room temperature to a solution of 210 g of NALCO™ 1034A silica and 400 g of isopropyl alcohol under rapid stirring. Stirring was continued for 15 minutes after the last addition, then the mixture was heated to 70° C. for 3 hours under continued rapid stirring. After heating, the solution was cooled to room temperature, and 63.6 g of ethoxylated pentaerythritol tetraacrylate (SR494) was added with constant stirring.

Next, most of the solvents were distilled off under reduced pressure using a rotary evaporator, leaving a white residue. The white residue was then redissolved in 150 g of isopropyl alcohol. All of the solvents were then removed using a rotary evaporator under reduced pressure, yielding a clear gel free liquid.

119.6 g of trimethylolpropane triglycidyl ether (ERISYS GE-30), 48 g of 1,4-butanediol diglycidyl ether (ERISYS GE-21, CVC Specialty Chemicals, Inc.; Moorestown, N.J.), and 12.4 g of polyethylene glycol (400) diacrylate (SR344) were added to the above clear liquid under ultrasonic agitation. Then 10.8 g of benzophenone, 5.6 g of 1-hydroxycyclohexyl phenyl ketone, 10.8 g of triarylsulfonium hexafluorophosphate salts-50% in propylene carbonate, and 0.9 g of FC 4430 (3M Surfactants) were added, and the mixture was continued under ultrasonic agitation for 3 hours. A clear coating was formed.

Film Formation & Testing

Films were formed and tested using the same method as used in Example 5.

Coating Performance

Table 9 shows the performance of the Example 9 Coating and an uncoated CR-39 substrate.

TABLE 9
Example 7 Results

| | | Uncoated |
|---|---|---|
| | Example 9 | CR-39 |
| Coating Thickness (μm) | 3-6 | n/a |
| Bayer Haze (%) | 13.2 | 25.4 |
| Bayer Ratio | 1.924 | 1 |
| Cross-Hatch Adhesion (PC)^{a} | poor | — |
| Cross-Hatch Adhesion (CR-39)^{b} | 100% Pass | — |
| Cross-Hatch Adhesion (Hardcoat)^{c} | 100% Pass | — |
| Cross-Hatch Adhesion (AR) | 100% Pass | — |
| Refractive Index | 1.51 | 1.49 |

^{a}On a bare polycarbonate substrate.
^{b}On a bare CR-39 substrate.
^{c}Commercially available polycarbonate and CR-39 substrates that were coated with thermal-cured hard coats.

As shown in Table 9, the Example 9 Coating exhibited a Bayer ratio of 1.924. This high Bayer ratio indicated that the coating was hard and abrasion resistant. This coating exhibited poor adhesion to bare polycarbonate substrate, but good adhesion to bare CR-39 substrate. Additionally, the coating showed good adhesion to CR-39 and polycarbonate substrates coated with thermal-cured hard coats. Thermal-cured hard coated lenses from Vision-Ease Lens, Inc. (poly-orcolite), Gentex Corporation (poly-Gentex), and Hoya Vision Care, N.A. (both polycarbonate and CR-39) lenses were evaluated and all exhibited good adhesion. Further, the Example 9 Coating was tintable.

The above written description sets forth the best mode of the invention, and describes the invention so as to enable a person skilled in the art to make and use the invention. The scope of the invention is not intended to be limited to the examples and may include other compositions that occur to those skilled in the art.

What is claimed is:

1. A UV curable coating composition comprising:
a) a colloidal silica which is surface-modified by a mixture of at least one vinyl silane and at least one acrylate silane;
b) at least one acrylic monomer;
c) at least one epoxy monomer; and
d) a catalytic amount of a UV polymerization initiator, the UV curable coating composition being essentially-free of water.

2. A UV curable coating composition as defined in claim 1, wherein the colloidal silica is derived from a water-based dispersion of colloidal silica.

3. A UV curable coating composition as defined in claim 1, wherein the at least one vinyl silane is vinyltrimethoxysilane and the at least one acrylate silane is 3-methacryloxypropyltrimethoxysilane.

4. A UV curable coating composition as defined in claim 3, wherein the ratio of vinyltrimethoxysilane to 3-methacryloxypropyltrimethoxysilane is between about 2 to about 1 and about 1 to about 2.

5. A UV curable coating composition as defined in claim 1, wherein the acrylic monomers are selected from the group consisting of ethoxylated pentaerythritol tetraacrylate, 1,6-hexanediol diacrylate, 2-hydroxyethyl acrylate, ethoxylated (6) trimethylolpropane triacrylate, tripropylene glycol diacrylate, polyethylene glycol (400) diacrylate, glycidyl methacrylate, and mixtures thereof.

6. A UV curable coating composition as defined in claim 1, wherein the epoxy monomers are selected from the group consisting of trimethylolpropane triglycidyl ether, 1,4 butanediol diglycidyl ether, and mixtures thereof.

7. A UV curable coating composition as defined in claim 1, wherein the UV polymerization initiator is selected from the group consisting of benzophenone, 1-hydroxycyclohexyl phenyl ketone, triarylsulfonium hexafluorophosphate salts, 2-hydroxy-2-methyl-1,4-(1-methylvinyl) phenyl propanone, 2-hydroxy-2-methyl-1-phenyl-1-propanone, and mixtures thereof.

8. A UV curable coating composition as defined in claim 1, further including a wetting agent.

9. A UV curable coating composition as defined in claim 8, wherein the wetting agent is selected from the group consisting of polyester modified polydimethylsiloxane, polyether modified polydimethylsiloxane, and mixtures thereof.

10. A cured coating composition made from the UV curable coating composition of claim 1.

11. A cured coating composition as defined in claim 10, further comprising an anti-reflective coating disposed on a surface of the cured coating.

12. A method for forming a UV curable coating composition comprising:
a) preparing an acrylate/vinyl silane-modified colloidal silica by mixing at least two silanes, one of which is an acrylate silane and the other which is a vinyl silane, with colloidal silica;
b) mixing at least one acrylic monomer with the acrylate/vinyl silane-modified colloidal silica in an aqueous/organic solvent system to form an acrylic-silica mixture;
c) removing solvent from the acrylic-silica mixture under vacuum until a gel begins to form;
d) adding a water miscible organic solvent to the acrylic-silica mixture to redisperse the mixture, including any gel formed, in solution;
e) removing the remaining solvent under vacuum to form a clear gel free liquid acrylic-silica mixture that is essentially free of water;
f) adding at least one multi-functional epoxy monomer and at least one acrylic monomer to the clear gel free liquid acrylic-silica mixture to form a UV-curable coating composition; and
g) adding a catalytic amount of a UV polymerization initiator to the UV-curable coating composition.

13. A method as defined in claim 12, further comprising the steps of:
h) distributing the UV curable coating composition on a substrate; and
i) curing the UV curable coating composition with UV radiation.

14. A method as defined in claim 12, wherein the acrylate/vinyl silane-modified colloidal silica is modified by an acrylate silane selected from the group consisting of 3-methacryloxypropyltrimethoxysilane, 3-acryloxypropyltrimethoxysilane, 2-methacryloxyethyltrimethoxysilane, 2-acryloxyethyltri-methoxysilane, 3-methacryloxy-propyltriethoxysilane, 3-acryloxypropyltriethoxysilane, 2-acryloxyethyltriethoxysilane and mixtures thereof.

15. A method as defined in claim 12, wherein the acrylate/vinyl silane-modified colloidal silica is modified by a vinyl silane selected from the group consisting of vinyltrimethoxysilane, ethylene vinylsilane, ethylene vinylacetate vinyl silane, and mixtures thereof.

16. A method as defined in claim 12, wherein the at least one acrylic monomer is selected from the group consisting of ethoxylated pentaerythritol tetraacrylate, 1,6-hexanediol diacrylate, 2-hydroxyethyl acrylate, ethoxylated (6) trimethylolpropane triacrylate, tripropylene glycol diacrylate, polyethylene glycol (400) diacrylate, glycidyl methacrylate, and mixtures thereof.

17. A method as defined in claim 12, wherein the at least one epoxy monomer is selected from the group consisting of trimethylolpropane triglycidyl ether, 1,4 butanediol diglycidyl ether, derivatives thereof, and mixtures thereof.

18. A method as defined in claim 12, wherein the water miscible organic solvent is selected from the group consisting of isopropyl alcohol, ethanol, butanol, and mixtures thereof.

19. A method as defined in claim 12, wherein the water miscible organic solvent is isopropyl alcohol.

20. A method as defined in claim 12, wherein the UV polymerization initiator is selected from the group consisting of benzophenone, 1-hydroxycyclohexyl phenyl ketone, triarylsulfonium hexafluorophosphate salts, 2-hydroxy-2-methyl-1,4-(1-methylvinyl) phenyl propanone, 2-hydroxy-2-methyl-1-phenyl-1-propanone, derivatives thereof, and mixtures thereof.

21. A method as defined in claim 12, wherein the vinyl silane is vinyltrimethoxysilane and the acrylate silane is 3-methacryloxypropyltrimethoxysilane.

22. A method as defined in claim 21, wherein the ratio of vinyltrimethoxysilane to 3-methacryloxypropyltrimethoxysilane is between about 2 to about 1 and about 1 to about 2.

23. A UV curable coating composition made by the method of claim 12.

24. A UV curable coating composition made by the method of claim 21.

25. A UV curable coating composition made by the method of claim 22.

26. A UV-cured coating composition made by the process of claim 13.

27. A UV curable coating composition comprising:
a) a vinyltrimethoxysilane and 3-methacryloxypropyltrimethoxysilane modified colloidal silica derived from a water based dispersion of colloidal silica;
b) at least one acrylic monomer;
c) at least one epoxy monomer;
d) a wetting agent; and
e) a catalytic amount of a UV polymerization initiator, the UV curable coating composition being essentially-free of water.

28. A UV curable coating composition as defined in claim 27, wherein the ratio of vinyltrimethoxysilane to 3-methacryloxypropyltrimethoxysilane is between about 2 to about 1 and about 1 to about 2.

29. A UV curable coating composition as defined in claim 27, wherein the at least one acrylic monomer is selected from the group consisting of ethoxylated pentaerythritol tetraacrylate, 1,6-hexanediol diacrylate, 2-hydroxyethyl acrylate, ethoxylated (6) trimethylolpropane triacrylate, tripropylene glycol diacrylate, polyethylene glycol (400) diacrylate, glycidyl methacrylate, and mixtures thereof.

30. A UV curable coating composition as defined in claim 27, wherein the at least one epoxy monomer is selected from the group consisting of trimethylolpropane triglycidyl ether, 1,4 butanediol diglycidyl ether, derivatives thereof, and mixtures thereof.

31. A UV curable coating composition as defined in claim 27, wherein the UV polymerization initiator is selected from the group consisting of benzophenone, 1-hydroxycyclohexyl phenyl ketone, triarylsulfonium hexafluorophosphate salts, 2-hydroxy-2-methyl-1,4-(1-methylvinyl) phenyl propanone, 2-hydroxy-2-methyl-1-phenyl-1-propanone, derivatives thereof, and mixtures thereof.

32. A UV curable coating composition as defined in claim 27, wherein the wetting agent is selected from the group consisting of polyester modified polydimethylsiloxane, polyether modified polydimethylsiloxane, and mixtures thereof.
